(19) United States
(12) Patent Application Publication
(10) Pub. No.: US 2015/0105865 A1
(43) Pub. Date: Apr. 16, 2015

(54) PROSTHETIC SOCKET WITH INTEGRATED COOLING CHANNELS

(71) Applicants: Brian Lawton Davis, Moreland Hills, OH (US); Christina Marie Webber, North Canton, OH (US)

(72) Inventors: Brian Lawton Davis, Moreland Hills, OH (US); Christina Marie Webber, North Canton, OH (US)

(21) Appl. No.: 14/511,659

(22) Filed: Oct. 10, 2014

Related U.S. Application Data
(60) Provisional application No. 61/889,079, filed on Oct. 10, 2013.

(51) Int. Cl. A61F 2/80

(52) U.S. Cl. CPC A61F 2/80; A61F 2002/501
USPC 623/33

(57) ABSTRACT

A residual limb prosthesis includes a socket for receiving a residual limb, the socket having a socket wall defining a limb-receiving surface. A coil-shaped channel extends through the socket wall from an inlet to an outlet, the outlet fluidly communicating with the ambient atmosphere. An air mover fluidly communicates with the inlet and the ambient atmosphere outside of the socket wall, the air mover drawing air from the ambient atmosphere and circulating the air through the coil-shaped channel to be exhausted at said outlet.

### CROSS-REFERENCE TO RELATED APPLICATIONS

The present application claims priority to U.S. Provisional Patent Application Ser. No. 61/889,079, filed Oct. 10, 2013, the entirety of which is incorporated herein by reference.

### BACKGROUND OF THE INVENTION

Many research efforts have been devoted to improving the foot, ankle, and knee units for transfemoral prosthetic components. While improving the mechanical functionality is critical, an equally important area of prosthetic design—the thermal environment—is often overlooked.

Current commercially available prosthetic liners and sockets are insulators, trapping heat around the residual limb. Therefore, an unnatural environment is created around the residual limb by this trapped heat, often causing the residual limb to sweat excessively leading to numerous dermatologic conditions, such as allergic contact dermatitis, microorganism infections, and verrucous hyperplasia. Atypical loading conditions experienced by a residual limb can exacerbate these skin problems, often reducing the quality of life for the amputee patient. Furthermore, amputee patient rehabilitation is hindered by the physical and psychological burdens caused by these conditions.

Efforts to improve the thermal properties of the prosthetic and socket would therefore benefit the amputee community.

The prosthetic environment is complex, making it difficult to model without making numerous assumptions and simplifications. Nevertheless, studies have been conducted to model residual limb temperature under various conditions. Some researchers have assumed heat generation by the residual limb to be uniform across the limb surface, however, this does not represent typical in vivo conditions. Furthermore, it is difficult to quantify a “cooling” effect and what it means to each individual amputee patient.

The prior art teaches a multilayered approach to analyze the prosthetic assembly, taking into account the thermal conductivity and thickness of each component, and wherein the effect of different prosthetic components on the temperature difference between the limb and environment was examined. This model assumed a uniform heat flux across the entire limb.

Further disclosures teach a 3-D finite element model of the limb and prosthetic socket to predict skin temperatures. In such models, elevated skin temperatures were observed over areas where muscles were present, even though muscles were not modeled individually. Decreased skin temperatures were found over bones and towards the distal end of the residual limb.

The present invention provides a design of a residual limb prosthesis which provides a normal thermal environment around the residual limb. The residual limb prosthesis includes a helical cooling channel within its socket wall manufactured from advances in 3D printing.

### SUMMARY OF THE INVENTION

In a first embodiment, the present invention provides a residual limb prosthesis comprising: a socket for receiving a residual limb, said socket having a socket wall defining a limb-receiving surface; a coil-shaped channel extending through said socket wall from an inlet to an outlet, said outlet fluidly communicating with the ambient atmosphere; and an air mover fluidly communicating with said inlet and said ambient atmosphere outside of said socket wall, said air mover operable to draw air from the ambient atmosphere and circulate the air through said coil-shaped channel to be exhausted at said outlet.

In a second embodiment, the present invention provides a residual limb prosthesis as in the first embodiment, wherein said socket is formed from additive manufacturing materials selected from the group consisting of plastic, metal alloys, titanium alloys, thermoplastic powders, epoxy materials or the like.

In a third embodiment, the present invention provides a residual limb prosthesis of as in either the first or second embodiment, wherein said inlet is positioned proximate the top of said socket and said outlet is positioned proximate an opposite end of the socket.

In a fourth embodiment, the present invention provides a residual limb prosthesis as in any of the first through third embodiments, wherein said inlet placed at the bottom of said socket and said outlet is placed at the opposite end.

In a fifth embodiment, the present invention provides a residual limb prosthesis as in any of the first through fourth embodiments, wherein said air mover is attached to said inlet, said air mover circulating ambient air through said coil-shaped channel.

In a sixth embodiment, the present invention provides a residual limb prosthesis as in any of the first through fifth embodiments, wherein said air mover is selected from the group consisting of a pump, a blower, a compressor and a fan.

In a seventh embodiment, the present invention provides a residual limb prosthesis as in any of the first through sixth embodiments, wherein said air mover is an air pump.

In an eighth embodiment, the present invention provides a residual limb prosthesis as in any of the first through seventh embodiments, wherein said socket is molded.

In a ninth embodiment, the present invention provides a residual limb prosthesis as in any of the first through eighth embodiments, wherein the socket is of one piece construction.

In a tenth embodiment, the present invention provides a residual limb prosthesis as in any of the first through ninth embodiments, wherein the socket is additive printed and said coil-shaped channel is defined by the absence of material forming the socket.

In an eleventh embodiment, the present invention a wearable structure comprising: a socket for being worn over a portion of an individual's anatomy, said socket having a socket wall defining a receptacle for the anatomy; a coil-shaped channel extending through said socket wall from an inlet to an outlet, said outlet fluidly communicating with the ambient atmosphere; and an air mover fluidly communicating with said inlet and said ambient atmosphere outside of said socket wall, said air mover operable to draw air from the ambient atmosphere and circulate the air through said coil-shaped channel to be exhausted at said outlet.

### BRIEF DESCRIPTION OF THE DRAWINGS

FIG. 1 is a perspective view of a residual limb prosthesis in accordance with the present invention;

FIG. 2 is a representative cross-sectional view of the residual limb prosthesis of FIG. 1, showing the coil-shaped channel of the residual limb prosthesis.

### DETAILED DESCRIPTION OF ILLUSTRATIVE EMBODIMENTS

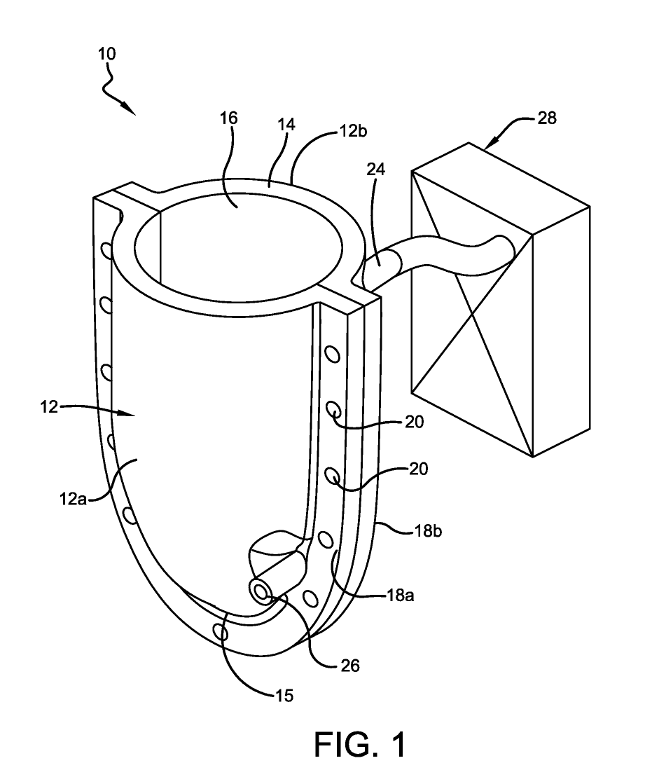

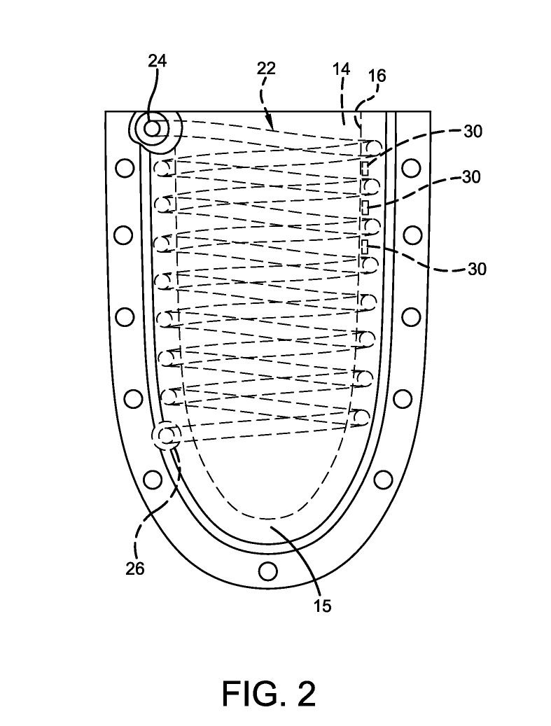

With reference to FIGS. 1-2, a residual limb prosthesis in accordance with this invention is shown and designated by the numeral 10. The residual limb prosthesis 10 includes a socket 12 for receiving a residual limb, wherein the socket 12 includes a socket wall 14 defining a limb-receiving surface 16. In some embodiments, the limb-receiving surface 16 is the inner surface of the socket wall 14, in order to come in direct contact with the limb in the socket 12. A liner, not shown but generally known in the art, might also be employed.

The residual limb prosthesis of the present invention further includes a coil-shaped channel 22 formed directly within the socket wall 14. The coil-shaped channel 22 extends throughout the socket wall 14 from an inlet 24 to an outlet 26, wherein the outlet 26 of the present invention fluidly communicates with the ambient atmosphere. The coil-shaped channel 22 is a continuous channel of any desired cross-sectional shape (though a circular shape will be practical). The coil-shaped channel 22 extends through the socket wall 14 to allow for the circulation of air through to cool the socket wall 14 and more particularly the residual limb retained in the socket wall 14. The coil-shaped channel 22 may extend through any section of the socket wall 14 that is desired to be cooled. In some embodiments, the coil-shaped channel 22 is designed to continuously extend from the top end to the bottom end of the socket 12. Although not shown in FIG. 2, it will be appreciated that the coil-shaped channel 22 can extend to the lowermost region 15 of the socket wall 14 to cool not only the sides of the residual limb but also the distal end thereof.

The coil-shaped channel 22 connects the inlet 24 to the outlet 26, wherein the outlet 26 fluidly communicates with the ambient atmosphere. In some embodiments, like that shown here, the inlet 24 is positioned proximate the top of the socket wall 14, and the outlet 26 is positioned below, preferably proximate the bottom of the socket wall 14. In other embodiments, the outlet 26 is positioned at the top end of the socket wall 14 and the inlet is positioned below, preferably proximate the bottom of the socket wall 14. The inlet 24 of the present invention is connected to an air mover 28. The air mover 28 fluidly communicates with the inlet 24 and the ambient atmosphere outside of the socket wall 14 and, when operational, serves to draw air from the ambient atmosphere and circulate it through the inlet 24 through the coil-shaped channel 22 to be exhausted at the outlet 26. In some embodiments, the air mover 28 is selected from a pump, a blower, a compressor and a fan. The air mover can be powered by battery or any other suitable source. In some instances, such as a prosthesis for a leg, the air mover might be powered by kinetic motion of the wearer. In some embodiments, the battery can be a Lithium-ion battery or the like.

In some embodiments, the socket wall 14 includes one or more heat sensors 30 positioned to monitor the temperature at the surface of the residual limb in the socket 12. The operation of the air mover 28 can be controlled as a function of the temperature monitored by the heat sensor(s) 30. Below an operational threshold temperature, the air mover 28 is in an off state, such that no air is circulated. Above the operational threshold temperature, the air mover 28 is placed in an on state such that air is circulated at a given rate. The rate at which the air is circulated can also be a function of the temperature monitored by the heat sensor(s) 30. As the temperature sensed by the heat sensor(s) 30 increases, the rate at which air is circulated can be increased to achieve more heat dissipation. The flow rate might be controlled stepwise, as various threshold temperatures are reached or it may be controlled in a more analog fashion.

In some embodiments, the air mover 28 moves the air through the coil-shaped channel at rates from 2.5 to 3 liters/minute, In other embodiments, the air mover 28 moves the air through the coil-shaped channel at rates from 2 to 2.5 liters/minute, In other embodiments, from 1 to 2 liters/minute, and in other embodiments, from 3 to 4 liters/minute.

In some embodiments, the socket wall 14 is formed through an additive manufacturing process (such as 3D printing) such that the coil-shaped channel 22 is formed as the socket wall 14 is formed. The coil-shaped channel 22 is thus defined simply by the absence of the socket wall material. Due to the fact that the socket 12 is not symmetrical, the additive manufacturing process will be found to be very useful. This will allow the socket 12 to be formed specifically to the exterior surface of a given residual limb. The socket 12 can be formed of any suitable material employed for additive manufacturing, as in the prior art or hereinafter developed. However, the suitable materials used for additive manufacturing need to be able to provide structural support for an amputee's limb. In some embodiments, the additive manufacturing materials can be selected from the group consisting of plastic, metal alloys, titanium alloys, thermoplastic powders, epoxy materials or the like.

In some embodiments, the material used to form the socket wall 14 is chosen to have a thermal conductivity from 0.10 W/m·K or more to 0.25 W/m·K or less. In other embodiments, the material used to form the socket wall 14 is chosen to have a thermal conductivity of from 0.15 W/m·K or more to 0.3 W/m·K or less, in other embodiments, from 0.3 W/m·K or more to 0.4 W/m·K or less, and in other embodiments when metallic alloys are employed, from 1 W/m·K or more to 300 W/m·K or less.

In FIGS. 1 and 2, more specifically described in the Experimental section below, the socket 12 is shown formed by securing two clamshell halves together, each half 12a, 12b having a flange 18a, 18b running along an outer edge and including a plurality of flange holes 20 that align to allow for securing together with fasteners (not shown).

Although a prosthesis is the focus herein, it will be appreciated that this concept could be employed for any wearable structure such as a helmet or a cast. Although air is a focus of a coolant material, other coolant materials could be used in a sealed system with an appropriate heat sink to cool the coolant after it is heated by traveling thought the coil-shaped channel. As alternatives to air, the coolant could be a liquid refrigerant or a phase change material.

### EXPERIMENTAL

### Methods

The temperature difference across the socket wall was observed in both computer simulations and bench-top testing for two proof-of-concept sockets. The first set of computer simulations were conducted using a steady temperature input on the inner socket wall to represent the residual limb. Residual limb temperature data from Peery et al. (2004) was used in the second set of computer simulations to represent a more clinically relevant scenario. Peery, J., Ledoux, W., Klute, G., 2004. Residual-limb skin temperature in transtibial sockets, Journal of Rehabilitation Research and Development 42, 147-154. The socket designs were then manufactured using 3D printing, and tested using a bench-top testing protocol. Detailed procedures for each of the above assessments of the socket design follow.

### Modeling: Steady Temperature Input

To determine if a cooling channel affects the temperature difference across the socket thickness, sockets were modeled using SolidWorks (Dassault Systemes, Waltham, Mass.). Both proof-of-concept sockets were modeled with the following scaled dimensions: 12.7 cm height, 1.0 cm wall thickness, and 7.6 cm outer wall diameter. Each socket model contained a cooling channel inlet port (such as inlet 24) and an outlet port (such as outlet 26). The final design for clinical use may have ports in different locations compared with the prototype. In preparation for bench-top testing, the sockets were designed with an outer flange (e.g., 18a, 18b), allowing for the socket to be constructed as two interlocking halves. Flange holes (e.g., 20) were included on the flange for the tightening of the socket halves together around a gasket. In a prosthetic system for clinical use, it is unlikely that the socket would be made of two separate halves. However, this approach allowed for easy cleaning of the cooling channel and straightforward assembly of the proof-of-concept socket.

Socket B contained a helical cooling channel 0.48 cm in diameter with eight revolutions (pitch=1.1 cm, 2.6° inward taper) (similar to that shown in FIG. 2). This was the maximum number of revolutions that could be included in the socket wall given the chosen dimensions. The Socket A did not contain a helical cooling channel. Steady-state thermal analyses were performed using ANSYS Workbench 14.5 (ANSYS, Cannonsburg, Pa.) to determine if a cooling channel affected the temperature difference between the inner and outer socket wall.

The socket material was assigned a thermal conductivity of 0.16 W/m·K based on average literature values for plastics typically used when making sockets (Bertels et al., 2011, Klittich et al., 2013, Klute et al., 2007). A prosthetic liner was not included in the model for this study. As such, it was assumed that the residual limb was in direct contact with the socket, so the inner wall of the socket was modeled as the same temperature of the residual limb skin. Based on the average of literature values of Peery et al., 2004, the limb was modeled as a steady 32.2° C. over a period of 28 minutes. Convection acted on the outer surface of the socket with an ambient room temperature of 21.9° C. For the modified prosthetic socket, the inner channel temperature was fixed at 21.9° C. to represent room temperature air flowing through the channel, corresponding to the conditions to be used in the bench-top testing.

The default mesh generator in ANSYS Workbench was used to mesh each socket using the medium smoothing feature, which was determined to be appropriate for use in the current study after conducting a convergence study. There was less than a 2% difference in the temperature differentials of the coarse, medium, and fine meshes tested in the convergence study. The mesh of Socket A contained 36,786 elements and 60,302 nodes. Socket B's mesh had 54,948 elements and 90,628 nodes. The simulation duration was 28 minutes, corresponding with the Peery et al. study. Time stepping was controlled using the automatic time stepping function in ANSYS.

Temperature profiles for each socket were generated. Inner and outer socket wall temperatures were recorded for proximal and distal locations on both sockets. The proximal temperatures were measured 4.1 cm from the top rim of the model sockets. This was the optimal location that was directly over a cooling channel. Furthermore, this location was 1 cm above the heating element that was to be used in the bench-top testing. The distal temperatures were measured 1 cm above the distal flange to avoid any influence of the flange on the temperature of the socket.

### Modeling: Clinical Temperature Input

The modeling procedure described above was repeated for both sockets using a dynamic and more physiologically relevant temperature input from Peery et al. (2004). For each socket, the simulation was run five times, corresponding to the temperature data from five patients collected by Peery et al. (2004). All other aspects of the simulation remained the same as in the steady temperature input simulation described previously. The temperature differentials for each patient (1-5), each socket (A and B1), and each location (proximal or distal) were analyzed for statistical significance (α=0.05) using Minitab 16 (Minitab, State College, Pa.) to perform an analysis of variance.

### Bench-top Testing

Prototype sockets were constructed from the SolidWorks™ models using an Eden 360 3D printer with VeroWhitePlus rigid opaque printing material (Stratasys, Eden Prairie, Minn.). This material was impermeable to water and mechanically suitable for the prototype sockets. The cooling channel was cleaned of the support material to allow for unobstructed flow through the channel. Two thermistors (Vernier, Beaverton, Oreg.) were affixed to the outside surface of the model sockets to monitor the temperature of the outer wall at distal and proximal locations. A third thermistor (Vernier, Beaverton, Oreg.) was affixed to the inner socket surface at the same height as the outer proximal sensor. The proximal temperatures were measured 4.1 cm from the top rim of the model sockets, directly above the cooling channel. This location was 1 cm above the heating element that was placed inside the water-filled socket. The distal temperatures were measured 1 cm away from the distal flange to avoid any influence of the flange on the temperature of the socket. These locations corresponded with the points used in the modeling simulations.

An air pump (Pacific Coast Distributing, Phoenix, Ariz.) with an output of 2.8 liters per minute was attached to the cooling channel inlet to circulate room temperature (21.9° C.) air through the cooling channel. Warm water at 32.2° C. was poured into the socket to mimic a uniform residual limb skin temperature. A small heater was used to maintain the water temperature at 32.2±0.3° C. to correspond with the average of the observed temperatures presented by Peery et al. A hand-wound, high temperature nickel wire was used for the heating element. The heater was controlled by an STC-1000 temperature controller (AGPtek, Brooklyn, N.Y.), allowing for a controlled release of heat into the system without overwhelming the small volume of water. A magnetic stir bar was used to mix the water inside of the socket so that the temperature of the water was uniform.

Temperature data was acquired from the thermistors for 30 minutes at 1 Hz using a LabQuest 2 unit (Vernier, Beaverton, Oreg.). Each socket was tested five times. Data were imported using LoggerPro 3 (Vernier, Beaverton, Oreg.), then the temperature differences between the inner and outer socket surface at the proximal and distal locations were calculated. An analysis of variance was performed in Minitab 16 (Mintab, State College, Pa.) to determine if a statistically significant (α=0.05) difference existed between the sockets.

### Results

Both bench-top testing and thermal simulations showed significant effects of the cooling channel.

### Modeling: Steady Temperature Input

Temperature profiles for the control and experimental socket that had an imposed steady inner temperature applied for 28 minutes showed cooling benefits through the wall of the socket. In both sockets, the maximum temperature reached on the inner socket wall was 32.2° C. The temperature differentials at the proximal and distal locations of Socket A were similar, 4.2° C. and 4.9° C. respectively. The proximal location of Socket B exhibited a greater temperature differential of 11.3° C. (Table 1).

TABLE 1

| | Proximal | Distal |
|---|---|---|
| Socket A | 4.2 | 4.9 |
| Socket B | 11.3 | 5.1 |

### Modeling: Clinical Temperature Input

The average temperature profiles of the proof-of-concept sockets using clinical temperature data showed a central portion in each socket that peaked at 33.1° C. The mean temperature difference between the inner and outer walls at the proximal end of the Socket A was 4.5° C., whereas at the distal end of Socket A, the mean temperature drop from the inner wall to the outer wall was 5.2° C. (Table 2).

TABLE 2

Temperature Difference (° C.)

| | Patient | Proximal | Distal |
|---|---|---|---|
| Socket A | 1 | 4.0 | 4.8 |
| | 2 | 4.3 | 5.1 |
| | 3 | 4.7 | 5.5 |
| | 4 | 5.4 | 6.4 |
| | 5 | 3.9 | 4.5 |
| | Average | 4.5 | 5.2 |
| Socket B | 1 | 10.1 | 5.0 |
| | 2 | 10.7 | 5.3 |
| | 3 | 11.8 | 5.8 |
| | 4 | 13.7 | 6.7 |
| | 5 | 9.4 | 4.7 |
| | Average | 11.1 | 5.5 |

At the proximal end of Socket B there was an 11.1° C. difference on average between the temperatures of the inner and outer walls. At the distal end of Socket B there was a 5.5° C. mean temperature difference. The analysis of variance showed that socket type (p=0.002) and the location on the socket (p=0.014) had a significant effect on the temperature differential across the socket wall.

### Bench-top Testing

Average temperature differentials of 5.6° C. and 6.2° C. were calculated for the proximal and distal ends of Socket A, the control socket. The eight spiral socket (Socket B) exhibited average temperature differentials of 6.4° C. and 6.7° C. at its proximal and distal ends, respectively (Table 3).

TABLE 3

| | Location | Predicted | Observed |
|---|---|---|---|
| Socket A | Proximal | 4.2 | 5.6 |
| | Distal | 4.9 | 6.2 |
| Socket B | Proximal | 11.3 | 6.4 |
| | Distal | 5.1 | 6.7 |

In the experimental testing, both socket design (p=0.001) and location on socket (p=0.006) had significant effects on the measured temperature difference.

### Discussion

A greater temperature drop across the socket wall suggests that the socket could draw heat away from the limb, towards the cooling channels. The proximal location on the modified socket exhibited a greater temperature difference between the inner and outer socket walls when compared to the control socket in both modeling simulations. Because the cooling channels are located only in the proximal portion of the socket, this finding was anticipated. The distal locations on both of the proof-of-concept sockets (channel and solid walled sockets) exhibited similar temperature differences between the inner and outer socket walls. In the models, the temperature on the outer socket surface was relatively constant directly above the cooling channels. This indicates that only one temperature reading from the outer surface was required to get a representative temperature difference across the socket wall where the cooling channel was located.

In the bench-top testing, the observed temperature differences between the inner and outer socket wall were of the same magnitude as the predicted values. Modifications to the socket had a significant (p=0.001) influence on the observed temperature differences. Socket B, containing the cooling channel, had the greatest temperature differences between its inner and outer socket walls in both the modeling and experimental aspects of this study. This indicated that heat was drawn towards the cooling channel from the warm inner surface of the socket. In the bench-top testing, it was difficult to compare the proximal and distal locations temperature differences because the absolute magnitudes of the values were so similar.

The computer simulations represented an idealized situation, where both socket inner wall temperature and the cooling channel temperature were uniform. While the bench-top testing included a stir bar to aid in homogenization of the water temperature in the socket, it may not have provided enough mixing to precisely match the assumption made in the model. The air flow through the channel was regulated by the capacity of the air pump, and could not be modified to determine the effects of different flow rates.

Efforts to aid in residual limb cooling would be beneficial to many amputee patients. Modifying the prosthetic socket, as presented above, could provide a possible solution to the problem of residual limb heating. The construction of a socket containing a helical cooling channel was only possible because of 3D printing. Three dimensional printing is a rapidly growing field with great potential for applications in the medical field, especially for custom made products like prosthetic sockets. For this study, VeroWhitePlus plastic (Stratasys, Eden Prairie, Minn.) was used to construct the sockets. This material performed well under testing and did not leak, warp, or melt. Other materials could be investigated to determine which has the optimum balance of thermal and mechanical properties for future use in 3D printing of prosthetic components.

When compared with the control socket, the modified socket containing an eight revolution cooling channel exhibited a greater temperature drop from the inner socket wall to the outer socket wall. Both socket type and location on the socket were statistically significant factors affecting the temperature difference between inner and outer socket walls. This finding was supported in both the modeling simulations and the bench-top testing. Thus, it was suggested that socket modifications could provide a significant cooling effect to a residual limb. This effect was observed even when only room temperature air flowed through the cooling channel in the experimental setup. As such, coolant management would not become an issue if the design concept was translated into a clinical application. Modified prosthetic sockets could be 3D printed in the future to help reestablish a normal thermal environment around the residual limb, thereby having a positive effect on many amputee patients' quality of life.

What is claimed is:

1. A residual limb prosthesis comprising:
a socket for receiving a residual limb, said socket having a socket wall defining a limb-receiving surface;
a coil-shaped channel extending through said socket wall from an inlet to an outlet, said outlet fluidly communicating with the ambient atmosphere; and
an air mover fluidly communicating with said inlet and said ambient atmosphere outside of said socket wall, said air mover operable to draw air from the ambient atmosphere and circulate the air through said coil-shaped channel to be exhausted at said outlet. 15

2. The residual limb prosthesis of claim 1, wherein said socket is formed from additive manufacturing materials selected from the group consisting of plastic, metal alloys, titanium alloys, thermoplastic powders, epoxy materials or the like.

3. The residual limb prosthesis of claim 1, wherein said inlet is positioned proximate the top of said socket and said outlet is positioned proximate an opposite end of the socket.

4. The residual limb prosthesis of claim 1, wherein said inlet placed at the bottom of said socket and said outlet is placed at the opposite end.

5. The residual limb prosthesis of claim 1, wherein said air mover is attached to said inlet, said air mover circulating ambient air through said coil-shaped channel.

6. The residual limb prosthesis of claim 5, wherein said air mover is selected from the group consisting of a pump, a blower, a compressor and a fan.

7. The residual limb prosthesis of claim 5, wherein said air mover is an air pump.

8. The residual limb prosthesis of claim 1, wherein said socket is molded.

9. The residual limb prosthesis of claim 1, wherein the socket is of one piece construction.

10. The residual limb prosthesis of claim 9, wherein the socket is additive printed and said coil-shaped channel is defined by the absence of material forming the socket.

11. A wearable structure comprising:
a socket for being worn over a portion of an individual's anatomy, said socket having a socket wall defining a receptacle for the anatomy;
a coil-shaped channel extending through said socket wall from an inlet to an outlet, said outlet fluidly communicating with the ambient atmosphere; and
an air mover fluidly communicating with said inlet and said ambient atmosphere outside of said socket wall, said air mover operable to draw air from the ambient atmosphere and circulate the air through said coil-shaped channel to be exhausted at said outlet.
